(19) United States
(12) Plant Patent Application Publication
(10) Pub. No.: US 2016/0174429 P1
(43) Pub. Date: Jun. 16, 2016

(54) Apple tree named '20/32/200'

(50) ;

(71) Applicant: BETTER3FRUIT, N.V., Heverlee (BE)

(72) Inventors: Inge De Wit, Lovenjoel (BE); Hendrik Eyssen, Holsbeek (BE); Johan Keulemans, Korbeek-Lo (BE); Johan Nicolai, Sint-Truiden (BE); Els Pauwels, Amougies (BE); Paul Van Laer, Heks (BE)

(73) Assignee: BETTER3FRUIT, N.V., Heverlee (BE)

(21) Appl. No.: 14/757,272

(22) Filed: Dec. 11, 2015

(30) Foreign Application Priority Data
Dec. 15, 2014 (QZ) 2014/3423

(51) Int. Cl. A01H 5/00

(52) U.S. Cl. PLT/162

(57) ABSTRACT

A new and distinct variety of Malus domestica apple tree named ‘20/32/200’, particularly characterized by scab resistance (monogenic Vf), well-balanced sweet-sour taste, juicy and crisp fruit texture, good storability and shelf life.

### LATIN NAME OF THE GENUS AND SPECIES OF THE PLANT CLAIMED

Malus domestica Borkh.

### VARIETY DENOMINATION

‘20/32/200’

### PRIORITY CLAIM

This application claims priority under 35 U.S.C. § 119(f) to European Community Plant Variety Office Application No. 2014/3423 filed Dec. 15, 2014.

### BACKGROUND OF THE INVENTION

The present invention relates to a new and distinct variety of Apple tree, botanically known as Malus domestica Borkh of the Rosaceae family, and hereinafter referred to by the variety denomination ‘20/32/200’.

The new Malus variety is a product of a controlled breeding program conducted by the inventors, Inge De Wit, Hendrik Eyssen, Johan Keulemans, Johan Nicola, Els Pauwels and Paul Van Laer in Belgium. The objective of the breeding program was to develop a new Malus variety with scab resistance, good taste and texture, as well as good storage capacity and shelf life.

The new Malus variety originated from a cross made by the inventors in 1990 in Belgium. The female or seed parent is the Malus domestica variety designated ‘DELCORF’ (registered, Plant Breeder's Rights No. FR 112364). The male or pollen parent is an unknown, proprietary variety of Malus domestica. The new Malus variety was discovered and selected by the inventors within the progeny of the stated cross in a controlled environment in 1996 in Belgium.

Asexual reproduction of the new Malus variety by grafting onto rootstocks was first performed in the winter of 1996-1997 in Belgium, and has demonstrated that the combination of characteristics as herein disclosed for the new variety are firmly fixed and retained through successive generations of asexual reproduction. The new variety reproduces true to type.

### BRIEF DESCRIPTION OF THE INVENTION

The following traits have been repeatedly observed and are determined to be unique characteristics of ‘20/32/200’ which in combination distinguish this Apple tree as a new and distinct variety:

1. scab resistance (monogenic Vf);

2. well-balanced sweet-sour taste;

3. juicy and crisp fruit texture;

4. good storability; and

5. good shelf life.

In comparison to the parental variety, ‘DELCORF’ (registered), ‘20/32/200’ differs primarily in the traits listed in Table 1.

TABLE 1

| | | Female Parent |
|---|---|---|
| | New Variety | ‘DELCORF’ |
| Trait | ‘20/32/200’ | (registered) |
| Fruit: pattern of over | flushed, striped and | weakly defined flush with |
| color | mottled | strongly defined stripes |
| Fruit: shape | obloid | globose conical |
| Fruit: time for harvest | 1 to 2 weeks after Elstar | 2 weeks before Elstar |
| Scab resistance | resistant | susceptible |

Of the many commercial varieties known to the present inventor(s), the most similar in comparison to the new Malus variety ‘20/32/200’ is the Malus variety ‘Topaz’ (registered, Plant Breeder's Rights No. CZ 322, DE APF 00111, CH 94-20-1033, EU 19951206, PL O S1, HU P9800450, SI MAD064, AR 7175, NZ APP149), in the following characteristics described in Table 2:

TABLE 2

| | | Comparison Variety |
|---|---|---|
| | New Variety | ‘TOPAZ’ |
| Characteristic | ‘20/32/200’ | (registered) |
| Fruit: pattern of over | flushed, striped | solid flush with strongly |
| colour | and mottled | defined stripes |
| Fruit: number of | many | medium |
| lenticels | | |
| Fruit: scarf skin | sometimes present | none |
| Fruit: taste | well-balanced sour-sweet | sour to very sour |

### BRIEF DESCRIPTION OF THE PHOTOGRAPHS

The accompanying photographs illustrate the overall appearance of the new Malus variety ‘20/32/200’ showing the colors as true as is reasonably possible with colored reproductions of this type. Colors in the photographs may differ slightly from the color values cited in the detailed botanical description, which accurately describe the color of ‘20/32/200’.

FIG. 1—shows a close-up view (side, top and bottom) of mature fruit of ‘20/32/200’.

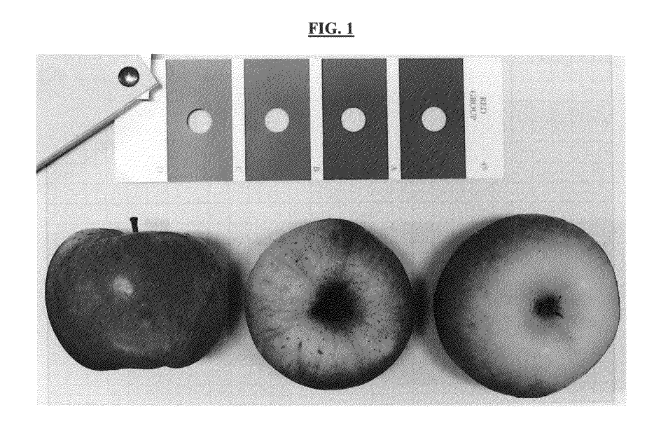

FIG. 2—shows dissection views of mature fruit of ‘20/32/200’.

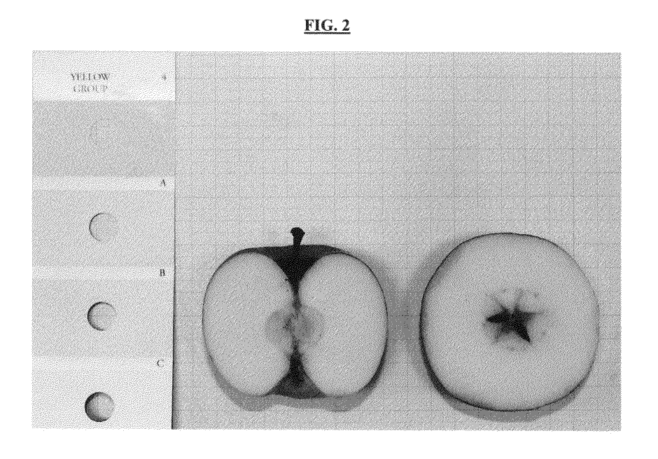

FIG. 3—shows a fruit-bearing tree of ‘20/32/200’, at 8 years of age.

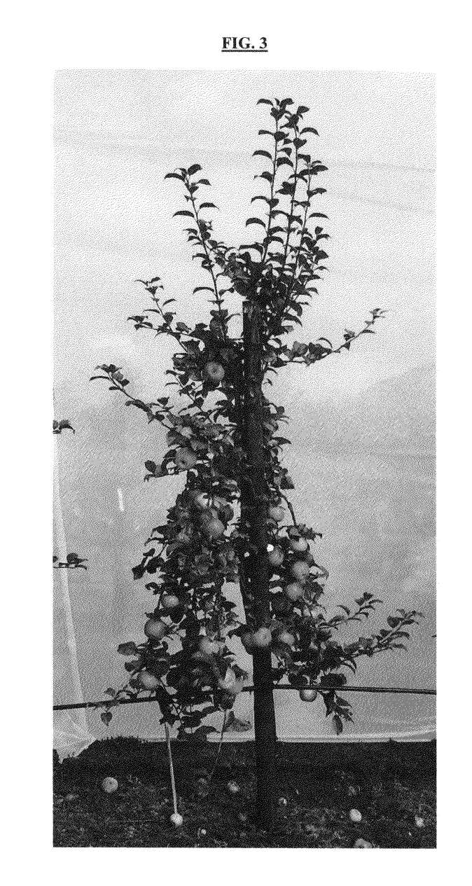

FIG. 4—shows a dormant tree of ‘20/32/200’, at 8 years of age

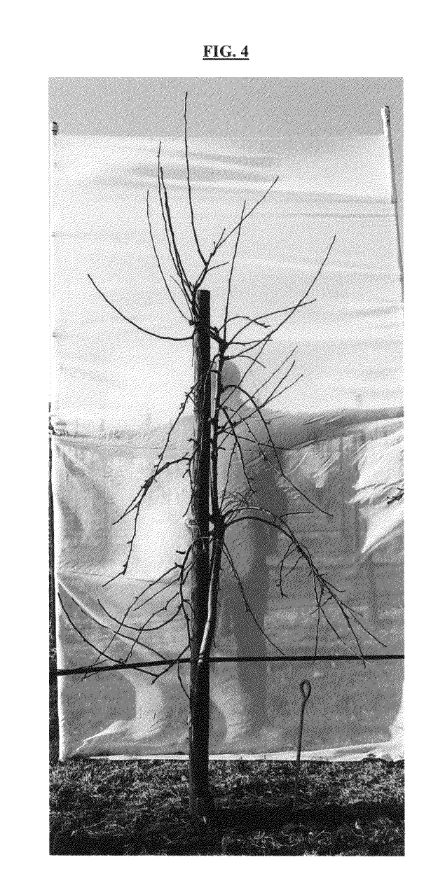

FIG. 5—shows a blooming tree of ‘20/32/200’, at 8 years of age.

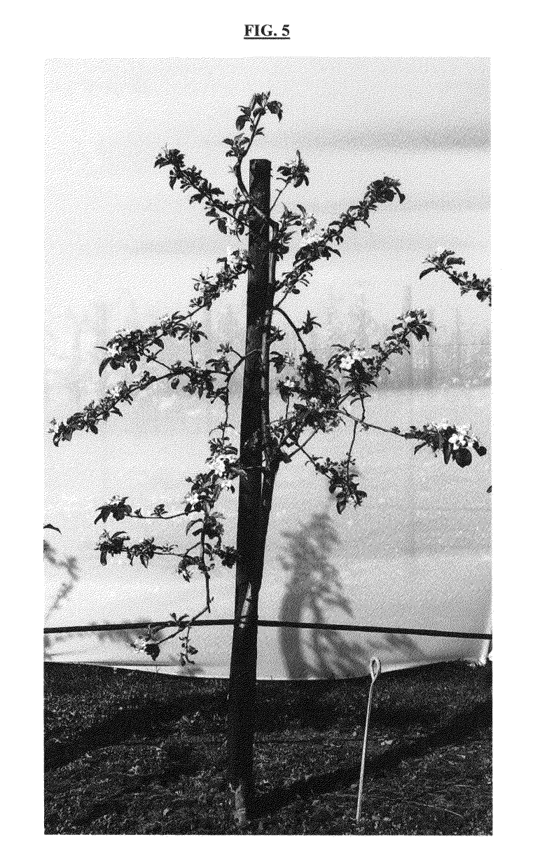

FIG. 6—shows inflorescence of ‘20/32/200’.

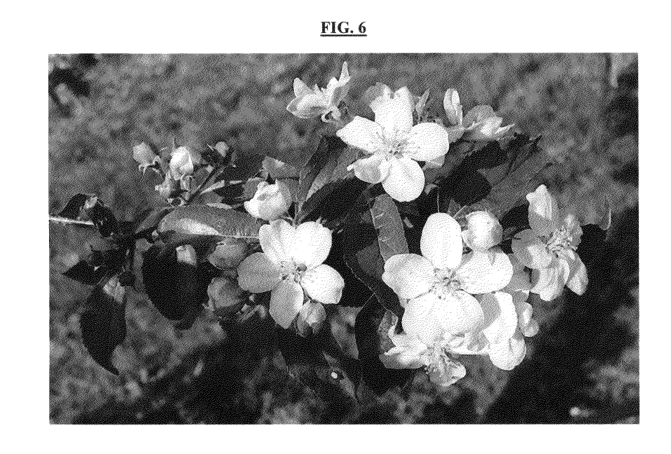

FIG. 7—shows the upper surface (top) and under surface (bottom) view of leaves of ‘20/32/200’.

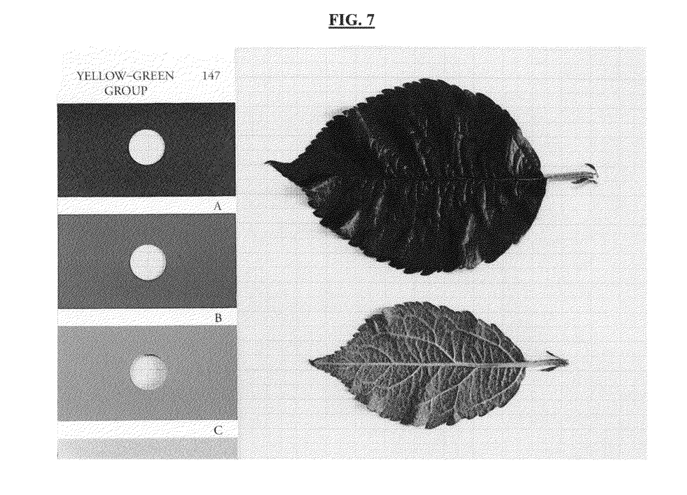

FIG. 8—shows different stages of development of the flowers and the (from left to right) top, side and bottom view of a fully expanded flower of ‘20/32/200’.

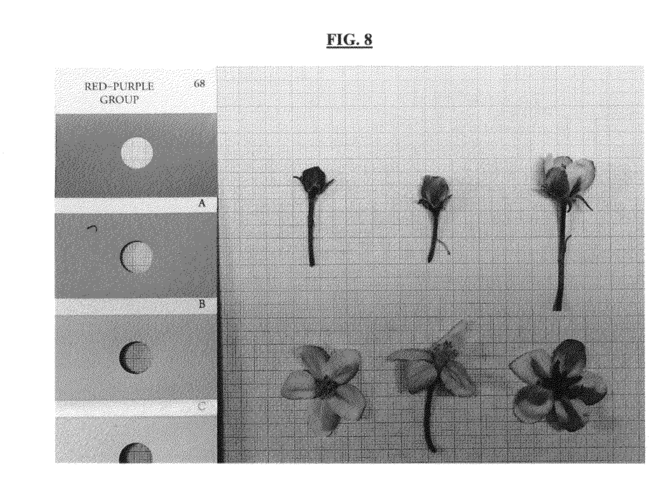

FIG. 9—shows different parts of a flower of ‘20/32/200’ (from left to right: upper and lower surface of petal, upper and lower surface of sepal, stamen, pistil).

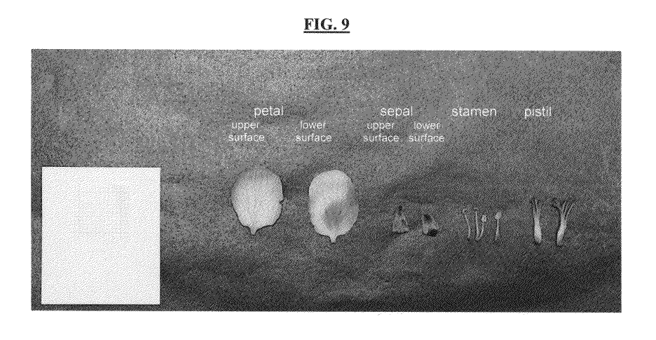

### DETAILED BOTANICAL DESCRIPTION

The new Malus variety ‘20/32/200’ has not been observed under all possible environmental conditions. The phenotype of the new variety may vary with variations in environment such as temperature, light intensity, and day length without any change in the genotype of the Apple tree.

The aforementioned photographs, together with the following observations, measurements and values describe trees of ‘20/32/200’ as grown in Rillaar, Belgium, under conditions which closely approximate those generally used in commercial practice.

Unless otherwise stated, the detailed botanical description includes observations, measurements and values based on 8 years old ‘20/32/200’ trees grown in Rillaar, Belgium from 2009 to 2015. Quantified measurements are expressed as an average of measurements taken from a number of trees of ‘20/32/200’. The measurements of any individual tree, or any group of trees, of the new variety may vary from the stated average.

Color references are made to The Royal Horticultural Society Colour Chart (R.H.S.), (4^{th} edition, 2001) , except where general colors of ordinary significance are used. Color values were taken under daylight conditions.

All of the trees of ‘20/32/200’, insofar as they have been observed, have been identical in all the characteristics described below.
Classification:
   Botanical.—Malus domestica.
Parentage:
   Female or seed parent.—Malus variety designated ‘DELCORF’ (registered).
   Male or pollen parent.—unknown, proprietary variety of Malus domestica.
Propagation: grafting onto rootstocks
Growing conditions: Light intensities: Full sunlight. Temperature: During day, grown in range of 2° C. to 23° C., and during evening, grow in range of −5° C. to 13° C. (based on average month temperature).
Tree:
   Age.—Observed trees were 8 years old.
   Vigor.—medium.
   Form.—ramified, drooping.
   Habit.—A medium-sized tree with one trunk and 5 +/−2 leaders; main branches drooping; crown symmetrical.
   Branching habit.—Main branches angle is 84 +/−10° with respect to trunk if allowed to grow naturally.
   Density.—about 2200 trees per hectare.
   Cropping behavior.—normal beginning production; low to medium productivity; low to regular flowering; biennial bearing.
   Type of bearing.—on spurs and long shoots.
   Production.—About 14.9 kg per tree in 2013.
   Size at maturity.—Height: About 215+/−7 cm Spread: About 121 +/−11 cm.
   Trunk.—Height (up to leaders): About 100 +/−10 cm Diameter: About 3.3 +/−0.2 cm Texture: Slightly rough Bark color: Primarily RHS 164A, with underbark RHS 156A Trunk Lenticels: Length: About 3.2 +/−0.6 mm Width: About 1.2 +/−0.3 mm Color: RHS 161B Density: About 3.6 +/−1.4 n° per cm².
   Branches.—Number per tree: About 33 +/−8 Length: maximum 67 +/−20 cm; minimum 5 +/−3 cm. Diameter (at 2 years): About 11 +/−3 mm. Surface texture: slightly rough Pubescence: none Color: Mature (after about 3 years old): RHS 166B New Growth: RHS 200B Internode length: About 31 +/−10 mm Internode diameter: About 6 +/−1 mm Branch lenticels: Length: About 2.9 +/−0.9 mm Width: About 1.2 +/−0.4 mm Color: RHS 161C Density: About 4.2 +/−1.9 n° per cm².
   Spur.—Present: Yes Distance between each spur: On the 2 and 3 year old branches, the distance is about 4 +/−2 cm Diameter of each spur: About 5 +/−1 mm Number of fruit per spur: About 0.8 +/−0.8.
Foliage:
   Arrangement.—Alternate, simple, petiolated.
   Lamina.—Size: Length: About 84 +/−13 mm (fully expanded leaf) Width: About 53 +/−12 mm (fully expanded leaf) Length/width ratio: 1.6 +/−0.1.
   Overall shape.—Obtuse but with a small pointy tip, petiolated Base shape: rounded Apex shape: acuminate.
   Margin.—serrate Texture: Upper surface: smooth, glabrous Under surface: smooth, medium pubescence (white) Attitude in relation to shoot: outwards Color (mature leaves): Upper surface: RHS 147A Under surface: RHS 138B Color (immature leaves): Upper surface: RHS 143A Under surface: RHS 138B.
   Venation.—Type: Pinnate Color: RHS 144D.
   Petiole.—Length: About 17 +/−4 mm Diameter: About 1.8 +/−0.3 mm Texture: smooth, high pubescence (white) Color: RHS 143C (upper surface), RHS 144D (lower surface), RHS 59B (at the petiole base, upper and lower surface).
   Stipule.—Arrangement: free standing Length (distance of stipules from basal attachment of petiole): About 8.3 +/−1.7 mm Width: About 1.6 +/−0.5 mm.
Inflorescence:
   Blooming time.—very early to early (after Ein Shemer, before Idared and Boskoop).
   Blooming period.—About 1 week.
   Fragrance.—quite strong.
   Type.—corymb.
   Number of flowers per inflorescence.—About 5.1 +/−0.8.
   Inflorescence size.—Diameter: About 80 +/−10 mm Depth: About 47 +/−7 mm.
   Buds.—Terminal Buds: Number per spur: About 1 Shape: a cone with an ellipsoidal base Length: About 6.8 +/−1.6 mm Width: About 4.2 +/−0.4 mm Texture: smooth, high pubescence Color: Apex RHS 200A, base RHS N186C Scales: Number: About 5.0 +/−1.1 Overall shape: triangular, folded double around bud Apex shape: acuminate or 3 sharp points Base shape: straight, fully grown together at base Color: Upper: RHS 187A Lower: RHS 144D Lateral Buds: Number per spur: About 3.1 +/−2.4 Shape: a cone with an ellipsoidal base Length: About 3.5 +/−0.9 mm Width: About 2.9 +/−0.5 mm Texture: smooth, slight pubescence at apex Color: RHS 187B (apex and base) Scales: Number: About 4.7 +/−1.1 Overall shape: triangular, folded double around bud Apex shape: acute Base shape: straight, fully grown together at base Color: Upper: RHS N186C Lower: RHS 144B.
   Petals.—Arrangement: overlapping Number per flower: 5 Size: Length: About 16 +/−2 mm Width: About 13 +/−1 mm Length/width ratio: 1.3 +/−0.2 Overall shape: ovate Apex shape: rounded Base shape: rounded Texture (upper surface): smooth, with hairs Texture (lower surface): smooth, no hairs Margin: entire Color (upper surface): Apex RHS N66D; base RHS N155B Color (lower surface): Apex RHS 68A; base RHS 69D.
   Sepals.—Number per flower: 5 Size: Length: About 8.6 +/−1.4 mm Width: About 4.2 +/−0.5 mm Length/width ratio: 2.1 +/−0.4 Overall shape: triangular Apex shape: aristate Base shape: truncate Texture (upper surface): smooth, hairy Texture (lower surface): smooth, slightly hairy Margin: entire Color (upper surface): Apex RHS 166A, base RHS 143C Color (lower surface): Apex RHS 166A, base RHS 143C.
   Pedicel.—Length: 32 +/−5 mm Diameter: 1.6 +/−0.2 mm Texture: smooth, hairy (white hairs) Color: RHS 144B.
Fruit:
   Keeping quality.—The fruit can be stored in cold temperature conditions for up to 2 months (normal atmosphere). It has a shelf life of about 2 weeks.
   Maturity when described.—Ripe for eating, 2 weeks storage.
   Maturity period after full bloom.—About 5 months after full bloom.
   Date of first and last picking (harvest).—About 2^{nd} and 3^{th} week of September.
   Type.—Pome.
   General shape.—obloid.
   Average weight.—About 171 +/−30 g.
   Fruit size.—Average height: About 61 +/−5 mm Average diameter (at widest point): About 77 +/−4 mm Position of maximum diameter: middle Height/thickness ratio: 0.80 +/−0.04.
   Stem.—Length: About 23 +/−7 mm Diameter: About 2.1 +/−0.2 mm Color: RHS 164A (full length, one side), RHS 144B (full length, one side), RHS 175B (at the end).
   Stalk cavity.—Depth: About 16.3 +/−1.9 mm Width: About 38.2 +/−8.3 mm.
   Eye basin.—Depth: About 9.9 +/−1.8 mm Width: About 36.9 +/−2.7 mm Crowning at calyx end: moderate Position of sepals: closed Calyx tube: partly open.
   Skin.—Thickness: 0.3 +/−0.1 mm Texture: tough Bloom: absent Greasiness: absent Firmness (at picking time): 6.1 +/−0.8 kg/cm² Overcolor color: RHS 45C and RHS 50B Percentage of skin surface with overcolor color: About 50% Pattern of overcolor: flushed, striped and mottled Intensity of overcolor: medium Ground color: RHS 150D and RHS 154D Skin Lenticels: Length: About 1.7 +/−0.4 mm Width: About 1.4 +/−0.3 mm Color: RHS 152C Density: About 3.3 +/−2.2 n° per cm².
   Flesh.—Color: RHS 4D Texture: crisp and juicy Aroma: moderate Eating quality: well-balanced sour-sweet Sugar content (at picking time): 13.3 +/−0.6 Brix Acidity/Starch (at picking time) 6.3 +/−0.2 g/1 malic acid Core: Symmetry of core: round Distinctness of core lines: medium Locules: Number (per fruit): 5 Length: About 8 +/−1 mm Width: About 3 +/−1 mm Form: teardrop shape.
Seeds: Number per fruit: About 6.5 +/−1.7 Number per locule: About 1.1 +/−0.7 Shape: teardrop shape Length: About 9.4 +/−0.6 mm Width: About 4.5 +/−0.4 mm Texture: smooth Color: RHS 175A.
Reproductive organs:
   Androecium.—Stamen: Number per flower: 18.6 +/−1.2 Length: About 7.2 +/−1.1 mm Anther: Length: About 2.2 +/−0.3 mm Color: RHS 11B Filaments: Length: About 5.8 +/−1.2 mm Color: RHS 155B Pollen: Amount: moderate Color: RHS 4A Pollination Requirements: cross pollination.
   Gynoecium.—Pistils: Quantity: 5 Length: About 11.9 +/−1.3 mm Stigmas: Length: About 1.3 +/−0.4 mm Color: RHS 144B Ovary: Length: About 4.9 +/−0.8 mm Width: About 1.8 +/−0.4 mm Color: RHS 144C.
Use: Fresh market.
Disease/pest resistance: resistant to scab (Venturia inaequalis, monogenic Vf).
Disease/pest susceptibility: tolerant to powdery mildew (Podosphaera inaequalis) and canker (Nectria galligena); medium susceptible to wooly apple aphid (Eriosoma lanigerum); susceptible to fire blight (Erwinia amylovora).
Winter hardiness: Tolerant to temperatures down to −10° C. without observed damage to wood and buds of dormant apple trees.
Drought/heat tolerance: Tolerant to temperatures up to 40° C., growth is limited by drought periods without irrigation.
Shipping/storage characteristics: not sensitive to bruising; good storability under ULO-conditions (maintains high quality for up to 5 months).

I claim:

1. A new and distinct variety of Malus domestica apple tree named ‘20/32/200’, as illustrated and described herein.
